(19) United States
(12) Patent Application Publication
(10) Pub. No.: US 2015/0037204 A1
(43) Pub. Date: Feb. 5, 2015

(54) METHOD AND DEVICE FOR THE GAS STERILIZATION OF PRODUCTS, WHEREIN THE PROCESS GASES ARE HOMOGENIZED BY A MIXER WITHOUT MOVABLE PARTS BEFORE BEING INTRODUCED INTO THE STERILIZATION CHAMBER

(71) Applicant: B. BRAUN MELSUNGEN AG, MELSUNGEN (DE)

(72) Inventors: Ralph Geiger, Felsberg (DE); Franz Fischer, Kassel (DE)

(21) Appl. No.: 14/379,898

(22) PCT Filed: Feb. 19, 2013
(86) PCT No.: PCT/EP2013/053285

§ 371 Date: Aug. 20, 2014

(30) Foreign Application Priority Data
Feb. 23, 2012 (EP) 12 156 678.0

(51) Int. Cl. A61L 2/20

(52) U.S. Cl. CPC A61L 2/20
USPC 422/26; 422/292; 422/28; 422/33; 422/34; 422/36

(57) ABSTRACT

Methods and devices for gas sterilization are described, in which products are sterilized using a sterilization gas. Process gases are mixed in a mixer without movable parts before being introduced into the sterilization chamber in order to obtain a homogenous gas mixture. A device for the gas sterilization of products comprises a mixer without movable parts and a sterilization chamber. The sterilization chamber has at least one inlet opening and at least one outlet opening. The mixer is arranged such that it is connected via a first line, which leads into a first opening in the mixer without movable parts, with the at least one outlet opening of the sterilization chamber and is connected via a second line, which leads into a second opening in the mixer without movable parts, with the at least one inlet opening of the sterilization chamber. A third gas supplying line leads into a third opening in the mixer.

### CROSS REFERENCE TO RELATED APPLICATIONS

This application is the U.S. national phase application of PCT International Application No. PCT/EP2013/053285 filed Feb. 19, 2013, which claims priority to European Patent Application No. EP 12156678.0 filed Feb. 23, 2012, the contents of each application being incorporated by reference herein.

### FIELD OF THE INVENTION

The present invention describes a method and a device for gas sterilization, in which products are sterilized using a sterilization gas. For this purpose, the process gases are mixed in a mixer without movable parts before being introduced into the sterilization chamber in order to obtain a homogenous gas mixture. In particular, the present invention relates to a device for the gas sterilization of products, comprising a mixer without movable parts and a sterilization chamber, wherein the sterilization chamber has at least one inlet opening and at least one outlet opening, and the mixer without movable parts is arranged such that it is connected via a first line, which leads into a first opening in the mixer without movable parts, with the at least one outlet opening of the sterilization chamber and is connected with a second line, which leads into a second opening in the mixer without movable parts, with the at least one inlet opening of the sterilization chamber and a third gas supplying line leads into a third opening in the mixer without movable parts.

### BACKGROUND

The sterilization with gases is a commonly used and well-known method. Especially prevalent is the use of ethylene oxide gas, which kills bacteria, mold and fungi and is therefore well suited for the sterilization of thermolabile substances. Because of its explosive and highly flammable properties, ethylene oxide (EO) is often mixed with inert gases, for example with carbon dioxide, nitrogen or with halocarbons. This can be done both during the filling of the gas into bottles (e.g. a mixture of 6% EO/94 CO₂) and in the sterilization chamber.

### DESCRIPTION OF THE RELATED ART

The usual method of the prior art provides that both the ethylene oxide and the inert gas are introduced into a sterilization chamber and are mixed with each other in there due to turbulences occurring during introduction of the process gases. This mixing is usually further supported by a ventilator which ensures circulation of the gas mixture during the entire sterilization cycle. Prior to the removal of the sterilization good from the chamber, the ethylene oxide is disposed and the sterilization good is usually cleaned from intercalated (diffused) ethylene oxide by repeated flushing with inert gas in order to protect the employee (explosion and also health protection).

There is little homogenization of the process gases due to the low pressure and the associated low density in the sterilization chamber and mainly occurs only outside of the products. This can cause that the process gases are not mixed properly and the distribution of the ethylene oxide in the sterilization chamber happens to be uneven. In the worst case, the ethylene oxide accumulates at the bottom of the sterilization chamber due to its higher density, which causes that the required concentrations for an effective sterilization in the individual regions—particularly at the upper part of the sterilization chamber—are not reached anymore. This is particularly critical for medical and medical-technical devices, in which an absolute sterility is indispensable and often leads to prolonged exposure times, which must be adhered to in order to guarantee the sterility of the products.

The use of ventilators that are designed to prevent exactly this is technically very complex and expensive because the ventilators have to satisfy the requirements of explosion protection. Alternatively it has been proposed to mix the process gases in a separate tank prior to the addition into the sterilization chamber. This approach is also associated with significant costs and extensive technical equipment and can only be integrated into existing systems with difficulties.

Sterilizations with other gases can also cause problems due to inhomogeneous distribution of the gas. In particular, the temperature distribution is a critical process parameter, for example during steam sterilization. In this type of sterilization, two phenomena are known among others which can lead to a disturbance of the process. On the one hand, inert gases that are introduced together with steam during the input of steam, form partial gas bubbles, which interfere with the steam condensation and thus the energy absorption of the sterilizing good. On the other hand, it can come to a partial overheating of strongly dried materials, particularly of textiles, due to hygroscopic condensation.

Accordingly, there is a need to improve the process of the gas sterilization in order to achieve a more homogeneous distribution of the gas mixture inside the sterilization chamber.

### SUMMARY OF THE INVENTION

Thus, an object of the present invention is to provide a method for the gas sterilization of products as well as a device by which a more homogeneous gas mixture can be produced for the gas sterilization.

It has surprisingly been found that this object is achieved by passing at least a portion of the gases during introduction into the sterilization chamber through a mixer without movable parts.

The inventive device for the gas sterilization of products comprises a sterilization chamber and a mixer without movable parts, which is connected with the sterilization chamber in such a way that at least a portion of the gases, which flow through the mixer without movable parts and are led into the sterilization chamber, is passed back from the sterilization chamber into the mixer without movable parts.

The present invention therefore relates to a device for the gas sterilization of products, comprising a mixer without movable parts (5) and a sterilization chamber (7), wherein the sterilization chamber (7) has at least one inlet opening and at least one outlet opening and the mixer without movable parts (5) is arranged such that it is connected via a first line, which leads into a first opening in the mixer without movable parts (5), with the at least one outlet opening of the sterilization chamber (7) and is connected with a second line, which leads into a second opening in the mixer without movable parts (5), with the at least one inlet opening of the sterilization chamber (7) and a third gas supplying line leads into a third opening in the mixer without movable parts (5).

In a preferred embodiment, the device further comprises a valve (1), wherein the valve (1) is arranged in the third gas supplying line to the mixer without movable parts (5).

Furthermore, devices are preferred, which further comprise another valve (3) instead of or together with the valve (1), wherein the valve (3) is arranged in the first line between the outlet opening of the sterilization chamber (7) and the mixer without movable parts (5).

In a further embodiment the mixer without movable parts further comprises an inlet for one or more process gases, an outlet for the input of the gases into the sterilization chamber and an inlet for process gases coming from the sterilization chamber.

Preferably, the inventive device for the gas sterilization of products comprises a sterilization chamber, which has at least one inlet opening and at least one outlet opening for gases, and a mixer without movable parts, wherein the mixer without movable parts is arranged such that at least a portion of the introduced gases flows first through the mixer without movable parts to be subsequently led via the at least one inlet opening into the sterilization chamber, wherein at the same time at least a portion of the gases, which is already in the sterilization chamber, is led from the sterilization chamber into the mixer without movable parts via the at least one outlet opening and mixes there with one or more of the introduced gases.

In a further embodiment, the mixer without movable parts comprises an inlet for one or more process gases and an outlet for the input of the gases into the sterilization chamber. In this embodiment, the inventive device for the gas sterilization of products comprises a sterilization chamber, which has at least one inlet opening and at least one outlet opening for gases, and a mixer without movable parts, wherein the mixer without movable parts is arranged such that at least a portion of the introduced gases flows first through the mixer without movable parts to be subsequently led via the at least one inlet opening into the sterilization chamber, wherein at the same time at least a portion of the gases, which is already in the sterilization chamber, is led via the at least one outlet opening from the sterilization chamber into a line supplying gas to the mixer without movable parts, in order to then mix with the introduced gas(es) in the mixer without movable parts.

With reference to FIG. 1, a particularly preferred embodiment of the device for the gas sterilization comprises a mixer without movable parts (5), valves (1, 3) and a sterilization chamber (7), wherein the sterilization chamber (7) has at least one inlet opening and at least one outlet opening and the mixer without movable parts (5) is arranged such that it is connected via a first line, which leads into a first opening in the mixer without movable parts (5), with the at least one outlet opening of the sterilization chamber (7) and is connected via a second line, which leads into a second opening in the mixer without movable parts (5), with the at least one inlet opening of the sterilization chamber (7) and a third gas supplying line leads into a third opening in the mixer without movable parts (5), wherein the valve (3) is arranged in the first line between the outlet opening of the sterilization chamber (7) and the mixer without movable parts (5) and the valve (1) is arranged in the third gas supplying line to the mixer without movable parts (5).

Preferably, the device for the gas sterilization further comprises a gas supplying bypass line with a valve (2), wherein the gas supplying bypass line is arranged such that it bridges the mixer without movable parts and directly leads into the sterilization chamber (7).

Traditionally, gas sterilization is also known under the term “chemical sterilization”, wherein advantage is taken here of the effect of toxic, volatile substances or gases. These include certain chemicals, such as for example formaldehyde, ethylene oxide, peracetic acid or hydrogen peroxide. For the present invention, any substance is in principle suitable for the gas sterilization, which is in the gaseous state or can be converted into the gaseous state and has a killing effect on bacteria, mold and/or fungi in that state. This includes in particular the sterilization with water vapor. Preferably, such a substance can be easily removed after sterilization or evaporates automatically.

The sterilization chamber can have any dimensions and can preferably be sealed tightly. Different parameters within the sterilization chamber can be regulated, such as for example the pressure and/or the temperature. The sterilizing good is placed in the sterilization chamber and the sterilization chamber is sealed in such a way that no gases can escape from the chamber uncontrolled. Preferably, the sterilizing good should be clean and dry and furthermore be preferably packed in special gas-permeable foils. Gas sterilization using chemical substances is generally used for thermolabile materials, wherein thermostable materials are preferably sterilized by water vapor sterilization.

The sterilization chamber has at least one inlet opening and an outlet opening for gases, wherein preferably a plurality of inlet openings and outlet openings may be present. Depending on the size and form of the sterilization chamber, desired sterilization time, the form of products and/or the sterilization gas used, a plurality of inlet and outlet openings may be present. Preferably, the inlet openings are located diagonally with respect to the outlet openings, so that no bypass occurs in the chamber.

In a further preferred embodiment, the gas is not simply introduced freely into the sterilization chamber, but it is spread in the sterilization chamber via one or more baffle plates, which are immovably attached inside the sterilization chamber.

According to aspects of the invention at least a portion of the gases or the sterilization gas is introduced via a mixer without movable parts into the sterilization chamber. Mixers without movable parts are wherein no movable elements for mixing are present in the interior of the mixer. Specifically, this means that mixing works without moving or movable parts and only the components to be mixed are moved or swirled. This, of course, does not rule out that, in general, movable parts are present on the mixer, however, these movable parts are not involved in the mixing of the gases.

As used herein, “mixer without movable parts” means that the internal parts of the mixer that come into contact with the gas are not movable. In other words, the “mixer without movable parts” has no parts, which cause by movement a mixing and/or transport of the gas. The term “mixer without movable parts” can therefore also be replaced in all the herein disclosed embodiments by the term “mixer without movable parts participating in the mixing” or “mixer without movable parts, which are responsible for the mixing” “mixer, wherein the mixing is not achieved by movable parts” or “mixer, that does not employ movable parts for the mixing”. Typical movable parts are propellers, movable nozzles, movable spirals, augers, impellers and other especially pivot-mounted parts. According to aspects of the invention, inside of the mixer, where the mixing of the gases takes place, no movable parts are present, which ensure the mixing or at least facilitate the mixing. In the device according to aspects of the invention the mixing of the gases within the mixer only takes place due to static obstacles and/or the deflection and/or turbulence of the gas flow. In addition, the mixing is improved by partly passing the gases from the sterilization chamber back into the mixer.

A mixer without movable parts for the mixing can therefore be referred to as a mixer with only static parts for mixing.

In other words, the present invention relates to a device for the gas sterilization of products, wherein the device comprises a mixer having only static parts (5) for mixing and a sterilization chamber (7), wherein the sterilization chamber (7) has at least one inlet opening and at least one outlet opening and the mixer with only static parts (5) is arranged such that it is connected via a first line, which leads into a first opening in the mixer with only static parts (5), with the at least one outlet opening of the sterilization chamber (7) and is connected with a second line, which leads into a second opening in the mixer with only static parts (5), with the at least one inlet opening of the sterilization chamber (7) and a third gas supplying line leads into a third opening in the mixer with only static parts (5).

Of course, this embodiment can also include one or both of the valves (1, 3). Thus, the present invention then relates to a device for the gas sterilization comprising a mixer with only static parts (5) for the mixing, valves (1, 3) and a sterilization chamber (7), wherein the sterilization chamber (7) has at least one inlet opening and at least one outlet opening and the mixer with only static parts (5) is arranged such that it is connected via a first line, which leads into a first opening in the mixer with only static parts (5), with the at least one outlet opening of the sterilization chamber (7) and is connected with a second line, which leads into a second opening in the mixer with only static parts (5), with the at least one inlet opening of the sterilization chamber (7) and a third gas supplying line leads into a third opening in the mixer with only static parts (5), wherein the valve (3) is arranged in the first line between the outlet opening of the sterilization chamber (7) and the mixer with only static parts (5) and the valve (1) is arranged in the third gas supplying line to the mixer with only static parts (5).

In a preferred embodiment, the mixer without movable parts is a jet pump. Preferably, the jet pump comprises a motive nozzle, a mixing chamber and a diffuser. In a further preferred embodiment, the jet pump comprises a motive nozzle, a mixing chamber, a diffuser, further an inlet for one or more process gases, an outlet for the input of the gases into the sterilization chamber and an inlet for process gases coming from the sterilization chamber.

In a further preferred embodiment, the mixer without movable parts is a static mixer. Static mixers are characterized in that guiding elements are arranged within the mixer such that the gas or the gases are mixed within a short flow path while flowing through the mixer. Herein, the guiding elements remain in a fixed position, i.e. are not movable and cause that the components are mixed through the flow cross-section.

According to aspects of the invention it is also possible not to use only one but also several mixers without movable parts. This is, among others, dependent on the process input parameters (e.g. pressure of the gases, flow rates). In a preferred embodiment, two mixers without movable parts are used, preferably three mixers without movable parts, further preferably four mixers without movable parts, and most preferably five mixers without movable parts. Here, combinations of different mixers without movable parts are possible. For example, a static mixer and a jet pump can be used simultaneously. Also possible is the use of mixers without movable parts, which consist of a combination of a static mixer and a jet pump. In such a case, the jet pump would be furthermore characterized in that it comprises guiding elements, similar to the guiding elements of a static mixer.

By the use of a mixer without movable parts in a device for the gas sterilization the sterilization process gets shorter, cheaper and more effective. Further, according to aspects of the invention no ventilator is required, by which the gases are swirled with each other in the sterilization chamber or during the introduction into the sterilization chamber. According to aspects of the invention the mixers without movable parts do not have any components for the mixing of gas, which effect the mixing of gas due to movement of these components, such as rotation.

### BRIEF DESCRIPTION OF THE DRAWINGS

The invention is best understood from the following detailed description when read in connection with the accompanying drawings. Included in the drawings are the following figures:

FIG. 1

Flow diagram of a mixer without movable parts in the piping system of a gas sterilization device.

FIG. 2

Schematic view of a mixer without movable parts in the form of a jet pump.

FIG. 3

Schematic representation of connection of a mixer without movable parts in the form of a jet pump to a sterilization chamber.

FIG. 4

Graph pressure profile during the sterilization process.

FIG. 5

Graph of temperature Min/Max values of three validation runs in a system without a mixer.

FIG. 6

Graph temperature Min/Max values of the three validation runs in a system with a mixer.

FIG. 7

Flow diagram of a mixer without movable parts in the piping system of a gas sterilization device that can be separated from the rest of the system.

FIG. 8

Graph of temperature profiles (mean process temperature deviation of all sensors) in a system with mixer without movable parts and a system with closed off mixer without movable parts.

FIG. 9

Graph of temperature profiles (mean process temperature of all sensors) in a system with mixer without movable parts and a system with closed off mixer without movable parts.

FIG. 10

Graph of temperature profiles (mean process temperature of all sensors) in a system with mixer without movable parts and a system with closed off mixer without movable parts within the area of the steam and ethylene oxide exposure time.

### DETAILED DESCRIPTION OF THE PREFERRED EMBODIMENTS

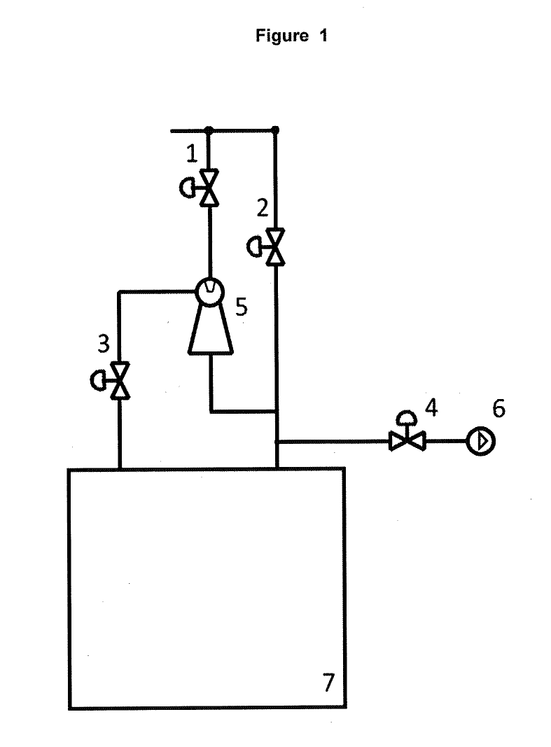

In FIG. 1 a mixer without movable parts in the piping system of a gas sterilization device is shown. The gas sterilization device consists of or comprises a mixer without movable parts 5, a sterilization chamber 7, an inlet valve 1, an inlet valve 2, an exhaust valve 4, as well as a vacuum pump 6 and a mixing valve 3. The input of the process gases, ethylene oxide, nitrogen and air, occurs preferably via the inlet valve 1 through the mixer without movable parts 5. Alternatively, the process gases may also be introduced through the valve 2 (bypass line) bypassing the mixer without movable parts 5. The air is sucked in from the environment, but can also be provided via a compressed-air line. The input of air occurs preferably via the valves 1 and 2. In the beginning, the ethylene oxide is present as liquid and is evaporated by a heat exchanger or evaporator (not shown). The ethylene oxide input occurs then preferably via the valve 1 as well as via the valve 2 in order to shorten the process time, but wherein the input can also be done only via the valve 1. The homogenization of the gas mixture prior to the ethylene oxide exposure time is preferably carried out by the subsequent nitrogen feed-in. The first input of nitrogen prior to the input of steam occurs preferably via the valves 1 and 2 (by-pass line and mixer). The second input of nitrogen directly after the ethylene oxide feed-in, occurs only through the mixer without movable parts 5. The subsequent nitrogen flushings can occur via both valves 1 and 2 (bypass line and mixer). The steam feed-in occurs preferably via the inlet valve 1 via the mixer without movable parts 5. Preferably, the mixer without movable parts 5 is mainly used for steam feed-in and nitrogen feed-in after the input of ethylene oxide.

It is apparent to those skilled in the art that the piping system and the valves may be differently arranged depending on the construction of the system. For example, it is possible that the line with the valve 4 branches directly off the sterilization chamber and not off the output line of the mixer without movable parts. These and other variations are within the skill of those of ordinary skill in the art and do not account for an inventive step.

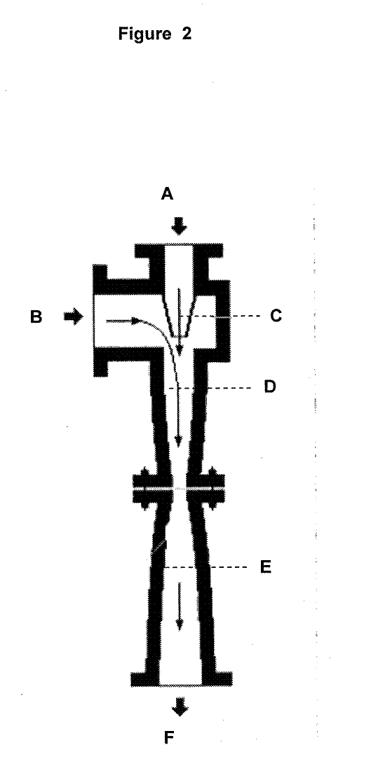

In FIG. 2 a mixer without movable parts in the form of a jet pump, as it can be used in the inventive device, is schematically illustrated. The mixer without movable parts according to this embodiment consists of a motive nozzle C, a mixing chamber D and a subsequent diffuser E. The process gases are introduced through the inlet A and the motive nozzle C into the mixing chamber D and the diffuser E. The input into the sterilization chamber occurs via the output F. A negative pressure is generated via the motive nozzle, which then withdraws process gases from the sterilization chamber through the inlet B. The mixing of the gases takes place directly behind the motive nozzle C in the mixing chamber D and the diffuser E. Due to the high gas velocities and the lower volume of the mixing chamber the mixing is significantly higher compared to the direct input into the sterilization chamber.

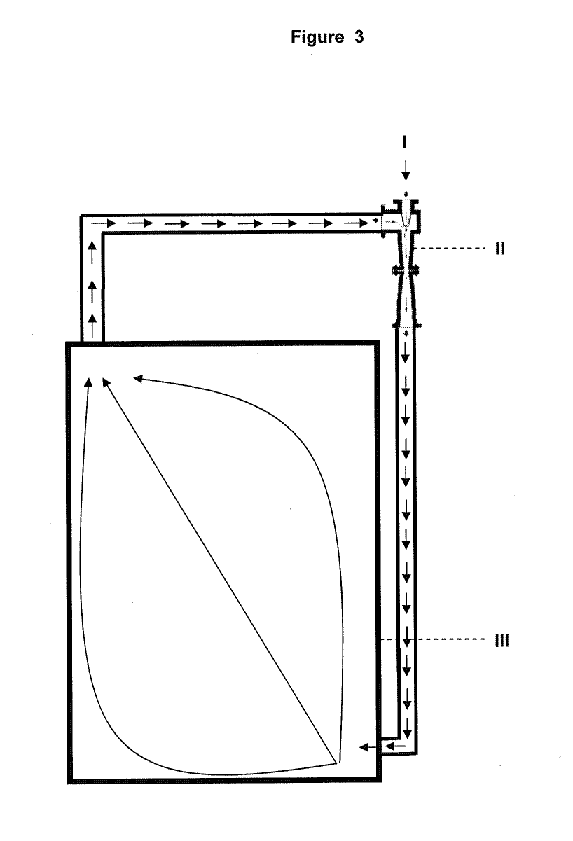

In FIG. 3, the connection of a mixer without movable parts in the form of a jet pump to a sterilization chamber is schematically illustrated. The gas input occurs, as already described, at point I in the mixer without movable parts in the form of a jet pump II. The circulation and mixing of the process gases occurs during the input by leading a pressurized gas (propellant flow) into the mixer without movable parts in the form of a jet pump. By momentum exchange, the gas from the sterilization chamber (suction flow) is sucked in, accelerated and compressed. The suction flow mixes with the very fast flowing propellant flow and is thereby accelerated. The inlet opening for the gases in the sterilization chamber III is arranged diagonal to the outlet opening, so that no bypass can be created in the chamber. Herein, the gases, which flow from the mixer without movable parts in the form of a jet pump via the inlet opening into the sterilization chamber, are sucked in again by the outlet opening preferably positioned diagonally opposite or as far away as possible. The input and distribution of the gases inside the chamber occurs via the pressure differential between the chamber and the input medium. The gas flow is arranged such that the sterilization goods are positioned in the flow or cause turbulences.

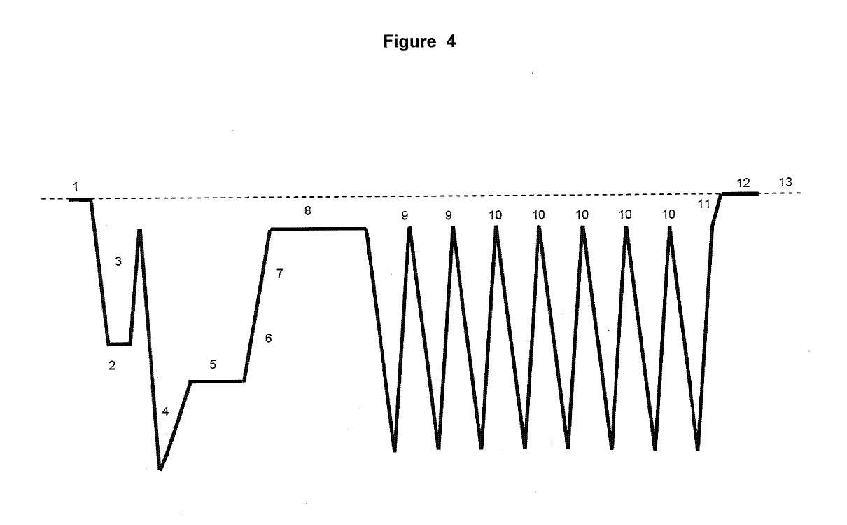

In FIG. 4, the pressure profile during the sterilization is shown schematically. The entire process is carried out at negative pressure, wherein during the process different gases are introduced and afterwards sucked off again from the sterilization chamber by a vacuum pump, wherein the suction of the gases is illustrated by the peaks directed downwards and the plateaus (steps 5 and 8) indicate, when the gases remain in the chamber. 1) The sterilization chamber is loaded and the process is started. 2) First, a first vacuum is applied and a leakage test is carried out, then 3) the input of nitrogen occurs with subsequent evacuation and a leakage test. 4) The input of steam and re-dosing with subsequent steam exposure time 5) follows. This is followed by the input of ethylene oxide 6), which is followed by the input of nitrogen 7). The actual sterilization is performed in step 8) during the exposure time of ethylene oxide. By the flushing steps 9) with nitrogen and 10) with air remaining ethylene oxide is removed. The entire process ends at the pressure equalization 11), and the discharge of the sterilized good 12).

The present invention also comprises a method for the gas sterilization of products in a sterilization chamber with at least one inlet and outlet opening and a mixer without movable parts, wherein at least a portion of the gases is mixed prior to or during addition into the sterilization chamber via a mixer without movable parts to a homogeneous gas mixture. A mixing before being introduced into the sterilization chamber in a mixer without movable parts has the further advantage that there is no resistance due to packing materials. By the mixing before being introduced into the sterilization chamber in a mixer without movable parts, interfering influencing variables, such as for example loading, chamber size, number of inlets or arrangement of the inlet points, are excluded. This increases the homogeneity and the comparability between unloaded chamber, partly loaded chamber, fully loaded chamber as well as small and large sterilization chambers.

In a further preferred embodiment, the method for the gas sterilization of products in a sterilization chamber with at least one inlet and outlet opening, and a mixer without movable parts, is characterized in that at least a portion of the gases is mixed in a mixer without movable parts during the addition into the sterilization chamber and subsequently led via an inlet opening into the sterilization chamber, wherein at the same time at least a portion of the gases, which is already in the sterilization chamber, is fed via an outlet opening out of the sterilization chamber into the mixer without movable parts and mixes there with one of the gases from the addition.

The addition is the gas, which is introduced for the first time in this cycle of mixing. This can be, for example, the input of inert gas, but also the addition of water vapor or the addition of the sterilization gas. Thus, addition does not mean the sucked gas from the sterilization chamber. The sucked gas from the sterilization chamber mixes with the gas of the addition in the mixer without movable parts and is led again into the sterilization chamber. This gas mixture is then sucked in again and mixes again with the gas of the addition in the mixer without movable parts.

In a preferred embodiment the method comprises the following steps:
a) applying a vacuum in the sterilization chamber,
b) inputting of inert gas into the sterilization chamber
c) inputting of water vapor into the sterilization chamber
d) inputting of the sterilization gas into the sterilization chamber,
e) inputting of inert gas into the sterilization chamber,
f) exposure time for the sterilization gas,
g) repeated flushing with inert gas and/or air,
h) pressure equalization;
   wherein the method is further characterized in that at least at one of the inputs b)-e) and/or g) the gas is mixed in a mixer without movable parts before being introduced into the sterilization chamber, and subsequently led via an inlet opening into the sterilization chamber, wherein at the same time at least a portion of the gases, which is already in the sterilization chamber, is fed via an outlet opening out of the sterilization chamber into the mixer without movable parts and mixes there with one of the gases from the inputs b)-e) and/or g).

Inert gases refer to gases that are very non-reactive (inert), i.e. participate only in a few and preferably in no chemical reactions, which could take place during the sterilization. Whether a particular gas for a particular application is referred to as an inert gas, is however still dependent on the specific case. For example, nitrogen and any noble gases (helium, neon, argon, krypton, xenon, radon) belong to the inert gases.

In principle, the method can be carried out with any type of sterilization gases. These include, for example, ethylene oxide or other alkylene oxides, formaldehyde, peracetic acid, hydrogen peroxide or other peroxides, and/or water vapor.

According to aspects of the invention, the method can also be applied to such gas sterilization methods, in which an inertization is not necessary. In such a case, the method comprises the following steps:
a) applying a vacuum in the sterilization chamber,
b) inputting of water vapor into the sterilization chamber,
c) inputting of the sterilization gas into the sterilization chamber,
d) exposure time for the sterilization gas,
e) repeated flushing with air,
f) pressure equalization;
   wherein the method is further characterized in that at least at one of the inputs b) and/or c) and/or e) the gas is mixed in a mixer without movable parts before being introduced into the sterilization chamber, and subsequently led via an inlet opening into the sterilization chamber, wherein at the same time at least a portion of the gases, which is already in the sterilization chamber, is fed via an outlet opening out of the sterilization chamber into the mixer without movable parts and mixes there with one of the gases from the inputs b) and/or c) and/or e).

### EXAMPLES

### Example 1

### Comparison of the Gas Sterilization Device with and without a Mixer without Movable Parts in the Form of a Jet Pump

The impact of a jet pump on the temperature distribution and thus also the gas distribution during sterilization with ethylene oxide was determined and compared with a device without a jet pump.

The ethylene oxide sterilization is a chemical sterilization, which is temperature dependent. An increase in the process temperature by 10° C. results in a doubling of the reaction rate or a halving of the exposure time. Therefore, the temperature is an important process parameter for the sterilization. In order to document differences in the effectiveness and homogeneity of the sterilization process, the recording of the temperature distribution during the exposure time of ethylene oxide was checked. For this purpose, temperature sensors were placed in the sterile good (pallets with disposable medical products) and the results were recorded. The recording of the temperature distribution was repeated three times to ensure the reproducibility of the results. Since the temperature feed occurs via the addition of water vapor, the temperature distribution may thus also be used as an indication of the gas distribution within the sterilization chamber and the sterile good.

System without Jet Pump:

Used were 43 temperature sensors type Data Trace, which were distributed in the palettes. Every minute one measuring point was recorded. For each series of measurements, the maximum value and minimum value of all 43 sensors was determined.

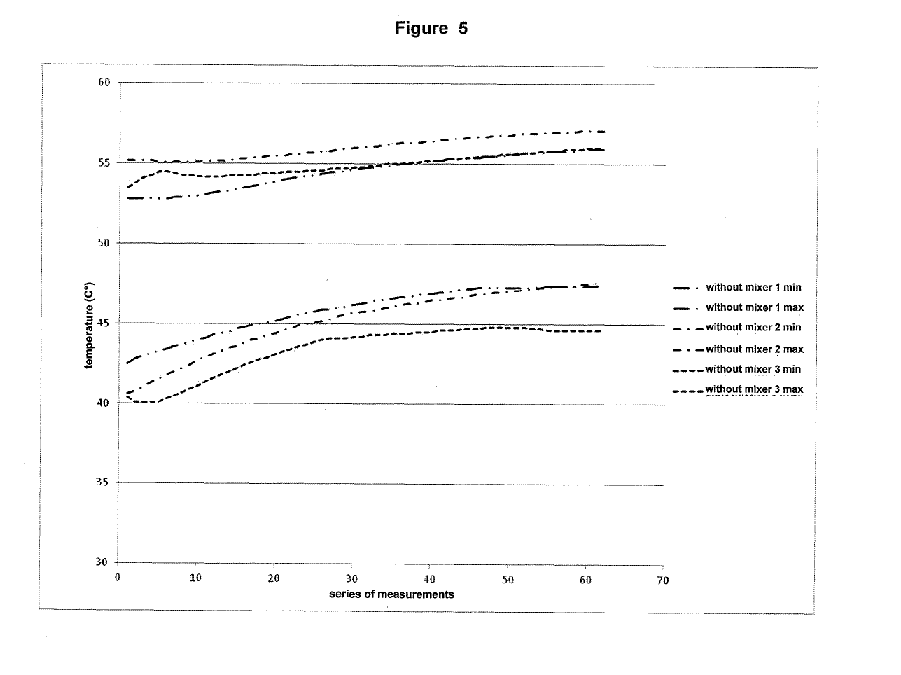

The results of the measurements in the system without a jet pump are shown in FIG. 5. FIG. 5 shows the min/max values of the three validation runs. For all series of measurements, the maximum temperature differences were determined during the exposure time. The result of the maximum temperature differences is illustrated in Table 1. The mean of all three runs results in a temperature deviation of 12.6° C. [(10.3+14.6+13.1)/3] between the maximum temperature and minimum temperature.

TABLE 1

| | | |
|---|---|---|
| Min (° C.) | Max (° C.) | Deviation |

Validation run 1
System without mixer

| | | |
|---|---|---|
| 42.50 | 52.80 | 10.30 |

Validation run 2
System without mixer

| | | |
|---|---|---|
| 40.60 | 55.20 | 14.60 |

Validation run 3
System without mixer

| | | |
|---|---|---|
| 40.40 | 53.50 | 13.10 |

System with Jet Pump:

Under similar conditions, the same process was carried out with a system, in which a jet pump was installed.

Used were 45 temperature sensors type Data Trace, which were distributed in the palettes. Every minute one measuring point was recorded. For each series of measurements, the maximum value and minimum value of all 45 sensors was determined.

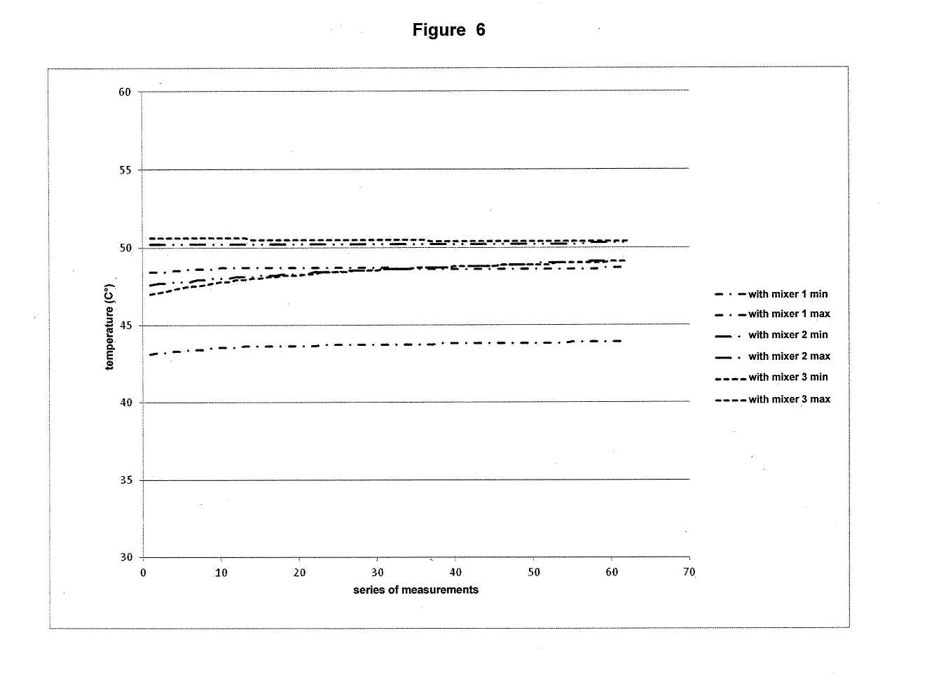

The results of the measurements in the system with a jet pump are shown in FIG. 6. FIG. 6 shows the min/max values of the three validation runs. For all series of measurements, the maximum temperature differences have been identified during the exposure time. The result of the maximum temperature differences is illustrated in Table 2. The mean of all three runs results in a temperature deviation of 3.8° C. [(5.3+2.6+3.6)/3] between the maximum temperature and minimum temperature.

TABLE 2

| | | |
|---|---|---|
| Min (° C.) | Max (° C.) | Deviation |

Validation run 1
System with jet pump

| | | |
|---|---|---|
| 43.10 | 48.40 | 5.30 |

Validation run 2
System with jet pump

| | | |
|---|---|---|
| 47.60 | 50.20 | 2.60 |

Validation run 3
System with jet pump

| | | |
|---|---|---|
| 47.00 | 50.60 | 3.60 |

### Comparability of the Results:

### Sensors:

In both validation studies (with and without jet pump), the same sensors were used.

### Process Parameters:

As explained above, the temperature was supplied to the product via the addition of steam. In both systems, the addition of steam took place via two inputs. After the addition the steam exposure time took place.

There, the process parameters were the following:

TABLE 3

| | | |
|---|---|---|
| Process parameter | Without jet pump | With jet pump |

| | | | | | |
|---|---|---|---|---|---|
| 1. | Input | | | | |
| | Start: | 50 | mbar | 50 | mbar |
| | End: | 125 | mbar | 125 | mbar |
| | Delta | 75 | mbar | 75 | mbar |
| 2. | Input | | | | |
| | Start: | 75 | mbar | 80 | mbar |
| | End: | 125 | mbar | 125 | mbar |
| | Delta | 50 | mbar | 45 | mbar |

| | | |
|---|---|---|
| Exposure time | 30 minutes at | 30 minutes at |
| | 120-125 mbar | 120-125 mbar |

The difference of 5 mbar in the second input of steam can be neglected in the overall process, or should even have a negative impact on the temperature distribution in the system with the jet pump, which was not the case. Thus, the process parameters of the two systems are comparable.

### Load:

All validation runs were performed with medical plastic disposables (syringes and IV-sets/infusion transition lines).
Load system without jet pump: 5 ml syringe (density 116 kg/m3, material PP, PE)
Load system with jet pump: IV-Sets (density 123-140 kg/m³, material PVC, PS)

The physical properties of the chamber-load are comparable with respect to temperature absorption. The difference in material and density can be neglected.

### Sterilization Chamber:

Both sterilization chambers are from the same supplier and the distribution of radiators is identical.

The sterilization chambers differ in their dimensions and in the design of the gas inlet openings.

TABLE 4

| | Without jet pump | With jet pump |
|---|---|---|
| Volume | 49.5 m³ | 55.1 m³ |
| Product volume | 30.6 m³ | 34 m³ |
| Steam inlets and | 9 inlet openings distrib- | 5 inlet openings on |
| arrangement | uted over both longitudinal | one side and 6 outlet |
| | sides of the chamber | openings opposite thereof |

Due to the different arrangements that are caused by the installation of the jet pump an effect cannot be ruled out. Therefore, a second series of experiments was performed in which a system has been installed, wherein the jet pump can be separated from the rest of the system by a slider or a valve.

### Example 2

### Comparison of the Gas Sterilization Device with Jet Pump that is Separable by a Slider from the Rest of the System

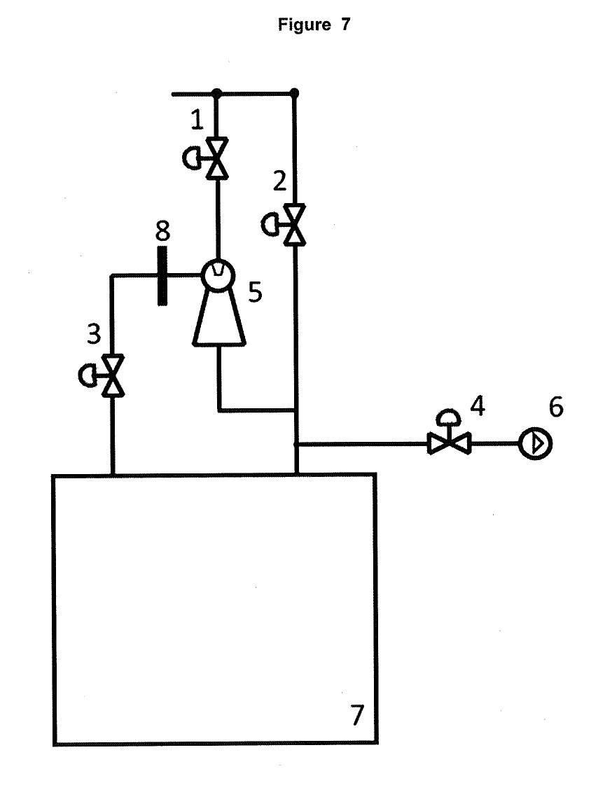

A validation run was carried out analogous to Example 1. In order to exclude the effect of the jet pump, a closable slider was installed between the access of the jet pump 5 and the valve 3 (cf. FIG. 7). Thereby the effect of the jet pump could be prevented, since no process gases could be sucked from the chamber and mixed in the mixing chamber.

### Comparison of the Temperature Deviations:

The measurement results of the temperature sensors were compared with the validation run of Example 1 (system with the jet pump). For this, the respective mean values of all temperature sensors at one point in time in the process were determined, and compared between the two test runs.

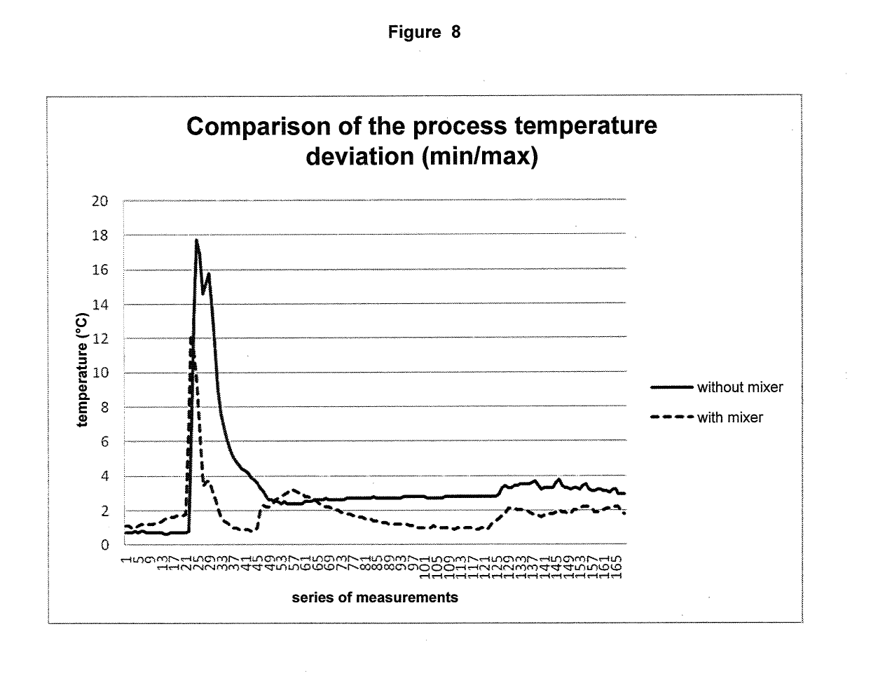

The two temperature profiles (mean process temperature deviation of all sensors) are shown in FIG. 8. As can be seen from FIG. 8, a more homogeneous distribution of the temperature is reached inside the load through the use of the mixer. The maximum deviation is reduced by 50% from 18° C. to 12° C. (cf. FIG. 8).

### Comparison of the Temperature Mean Values:

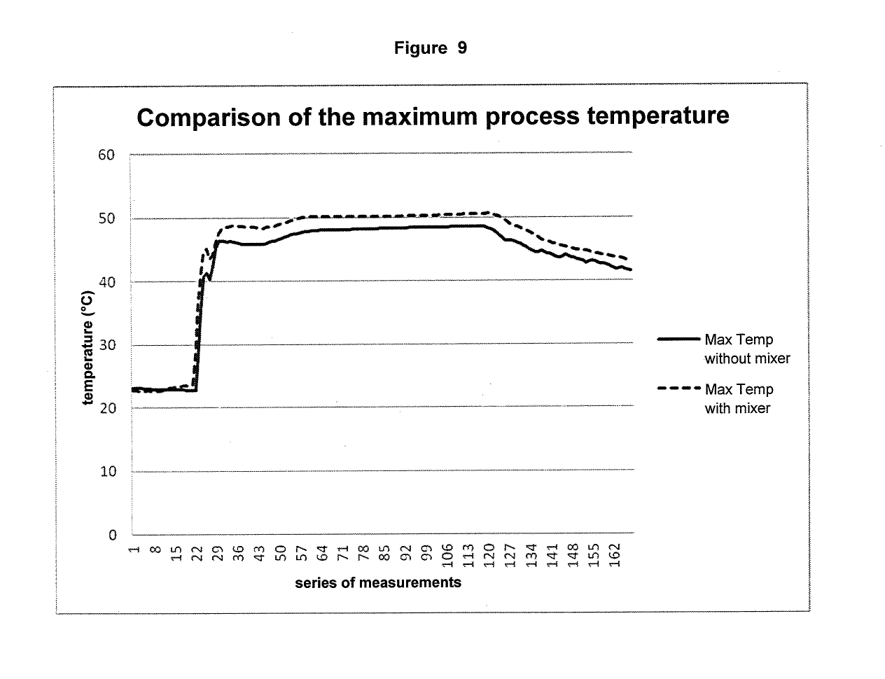

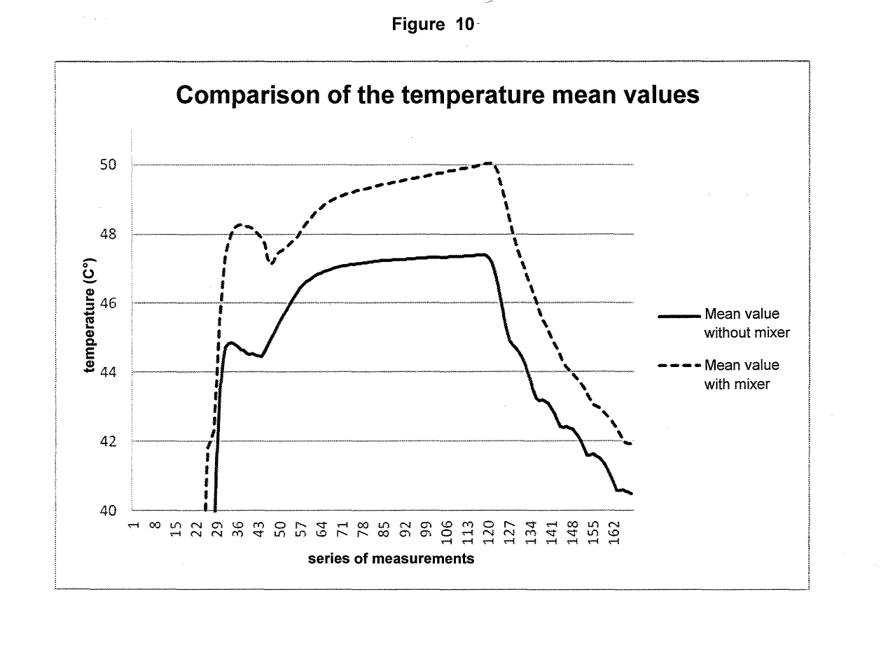

Furthermore, the mean process temperature of all temperature sensors was determined at all points in time during the process and compared with each other. The result of this comparison is shown in FIG. 9. It has been found that the mean process temperature (mean value of all sensors at one point in time) increases significantly by the use of the jet pump with otherwise identical process conditions. In FIG. 10 again a comparison of the temperature mean values for the range of the particularly important steam and ethylene oxide exposure times is shown. Especially here, a clear increase of the mean temperature in the system with the jet pump is shown.

Thus, it shall be noted that the improvement of the temperature distribution in the sterilization chamber is clearly attributable to the use of the jet pump. The mixing of the process gases prior to the actual input into the sterilization chamber results in a more homogeneous gas mixture, which is recognizable by the more uniform temperature distribution.

1-13. (canceled)

14. Device for the gas sterilization of products, comprising a mixer without movable parts and a sterilization chamber, wherein the sterilization chamber has at least one inlet opening and at least one outlet opening and the mixer without movable parts is arranged such that it is connected via a first line, which leads into a first opening in the mixer without movable parts, with the at least one outlet opening of the sterilization chamber and is connected with a second line, which leads into a second opening in the mixer without movable parts, with the at least one inlet opening of the sterilization chamber; and wherein a third gas supplying line leads into a third opening in the mixer without movable parts.

15. Device according to claim 14, further comprising a valve, wherein the valve is arranged in the third gas supplying line to the mixer without movable parts.

16. Device according to claim 14, further comprising a valve, wherein the valve is arranged in the first line between the outlet opening of the sterilization chamber and the mixer without movable parts.

17. Device according to claim 14, further comprising a first valve and a second valve, wherein the first valve is arranged in the first line between the outlet opening of the sterilization chamber and the mixer without movable parts and the second valve is arranged in the third gas supplying line that leads to the mixer without movable parts.

18. Device according to claim 14, wherein the mixer without movable parts has a first inlet for one or more process gases, an outlet for the input of the gases into the sterilization chamber, and a second inlet for process gases coming from the sterilization chamber.

19. Device according to claim 14, wherein the mixer without movable parts includes a jet pump, which comprises a motive nozzle, a mixing chamber and a diffuser.

20. Device according to claim 14, wherein the mixer without movable parts includes a static mixer.

21. Device according to claim 14, further comprising a gas supplying bypass line with a valve, wherein the gas supplying bypass line is arranged such that it bridges the mixer without movable parts and directly leads into the sterilization chamber.

22. Method for the sterilization of products with process gases in a sterilization chamber including at least one inlet opening and at least one outlet opening, the method comprising mixing at least a portion of the process gases into a homogeneous sterilization gas via a mixer without movable parts; and adding the homogeneous sterilization gas into the sterilization chamber.

23. Method according to claim 22, wherein the portion of the process gases is mixed in the mixer without movable parts before being introduced into the sterilization chamber via the inlet opening into the sterilization chamber, wherein at the same time at least a portion of process gases already in the sterilization chamber is fed via the outlet opening out of the sterilization chamber into the mixer without movable parts.

24. Method according to claim 22, comprising the following steps:
a) applying a vacuum in the sterilization chamber,
b) inputting of inert gas into the sterilization chamber,
c) inputting of water vapor into the sterilization chamber,
d) inputting of the sterilization gas into the sterilization chamber,
e) inputting of inert gas into the sterilization chamber,
f) exposure time for the sterilization gas,
g) repeated flushing with inert gas and/or air, and
h) pressure equalization;
wherein at least at one of the inputting steps b)-e) or g) the process gases are mixed in the mixer without movable parts before being introduced into the sterilization chamber, and subsequently led via at least one inlet opening into the sterilization chamber, wherein at the same time at least a portion of process gases already in the sterilization chamber is fed via at least one outlet opening out of the sterilization chamber into the mixer without movable parts and mixes there with at least one of the gases from at least one of the inputting steps b)-e) or g).

25. Method according to claim 22, comprising the following steps:
a) applying a vacuum in the sterilization chamber,
b) inputting of water vapor into the sterilization chamber,
c) inputting of the sterilization gas into the sterilization chamber,
d) exposure time for the sterilization gas,
e) repeated flushing with air, and
f) pressure equalization;
wherein at least at one of the inputting steps b), c), or e) the gas is mixed in the mixer without movable parts before being introduced into the sterilization chamber, and subsequently led via an inlet opening into the sterilization chamber, wherein at the same time at least a portion of process gases already in the sterilization chamber is fed via an outlet opening out of the sterilization chamber into the mixer without movable parts and mixes there with at least one of the gases from at least one of the inputting steps b), c), or e).

26. Method according to claim 22, wherein the sterilization gas is at least one gas selected from the group consisting of ethylene oxide, formaldehyde, peracetic acid, hydrogen peroxide and water vapor.
